(19) United States
(12) Patent Application Publication
(10) Pub. No.: US 2010/0204720 A1
(43) Pub. Date: Aug. 12, 2010

(54) Device for Fitting a Cutting Blade Holder of a Microsurgical Cutting Instrument, in particular a Cutting Instrument for use in Refractive Eye Treatments, with a Cutting Blade Unit

(75) Inventors: Tobias Jeglorz, Nurnberg (DE); Christof Donitzky, Eckental (DE)

Correspondence Address: HAYNES AND BOONE, LLP;IP Section, 2323 Victory Avenue, Suite 700, Dallas, TX, 75219, US

(73) Assignee: Wavelight AG, Erlangen (DE)

(21) Appl. No.: 12/063,586

(22) PCT Filed: Aug. 3, 2006
(86) PCT No.: PCT/EP06/07699

§ 371 Date: Apr. 30, 2010

(30) Foreign Application Priority Data
Aug. 12, 2005 (EP) 05017649.4

(51) Int. Cl. A61F 9/013; A61F 9/007

(52) U.S. Cl. 606/166

(57) ABSTRACT

A device (10) for fitting a cutting head (26) of a microsurgical cutting instrument, in particular a cutting instrument for use in refractive eye treatments, with a cutting blade unit, comprises a blade cartridge (12) which can be detachably coupled to the cutting blade holder (26) and in which a cutting blade unit is received. In addition, the fitting device comprises a slide (20) which is or can be held slidably relative to the cartridge and by means of which the cutting blade unit can be pushed out of the blade cartridge into a blade receiving pocket of the cutting head (26). Preferably, the slide (20) can be coupled to the cutting blade unit not only in a thrust-proof manner, but also in a traction-proof manner, so that after use the cutting blade unit can be withdrawn from the cutting head again by means of the slide (20) and guided back into the cartridge (12).

The invention relates to a device for fitting a cutting blade holder of a microsurgical cutting instrument, in particular a cutting instrument for use in refractive eye treatments, with a cutting blade unit.

For the treatment of refractive sight defects of the human eye, it is known to use a microsurgical cutting instrument called a microkeratome to cut a slice (flap) from the cornea surface in such a way that it is still connected to the cornea on one side (hinge). By lifting the flap and folding it away, the cornea interface (stroma) becomes accessible for reshaping of the cornea. The reshaping is carried out typically by means of an excimer laser (e.g. 193 nm) which suitably ablates the exposed cornea regions. After completion of the laser treatment, the flap is folded back.

The microkeratome normally has a suction ring unit which is to be placed on the eyeball and on which a cutting head (cutting blade holder) fitted with a cutting blade unit is held. By application of a vacuum, the suction ring unit is firmly sucked onto the eyeball (limbus) and thus fixes the latter. The cutting head is movably guided on the suction ring unit and can be moved over the cornea by means of an electric-motor drive unit. In the process, a cutting blade of the cutting blade unit cuts into the cornea and cuts the flap. During the forward stroke of the cutting head, the cutting blade unit is normally excited to oscillate laterally.

For reasons of hygiene, it is customary to replace the cutting blade unit after each operation. The insertion of a fresh cutting blade unit into the cutting head has hitherto been carried out mostly by introducing the cutting blade unit directly by hand or with the aid of tweezers into a receiving pocket, provided on the cutting head, for the cutting blade unit. After use, the cutting blade unit is withdrawn from the cutting head likewise with one's bare hand or with the aid of tweezers and then disposed of. It can readily be understood that there is a very high risk of injury in this process (the cutting blades are extremely sharp) and contamination of unprotected hands. The cutting blade for its part may be contaminated by hand injuries, which is naturally to be avoided in the case of a fresh cutting blade. There is also a risk of the cutting blade, in particular its cutting edge, being damaged by improper handling. Such damage of the cutting blade may be scarcely noticeable and yet greatly affect the cutting result. Since the cutting blade and the cutting head are high-precision components, a trained eye and great fine motor activity are required for the insertion and removal of the cutting blade unit, in order not to jeopardise the correct use of the microkeratome.

The object of the invention is to provide a device which permits simple, safe and damage-free insertion of a cutting blade unit into a cutting blade holder of a microsurgical cutting instrument, in particular for use in ophthalmic surgical treatments.

To achieve this object, such a device comprises, according to the invention, a blade cartridge which can be detachably coupled to the cutting blade holder and in which at least one cutting blade unit is received, and a slide which is or can be held slidably relative to the blade cartridge and by means of which the cutting blade unit can be pushed out of the blade cartridge into a blade receptacle of the cutting blade holder. The solution according to the invention makes it possible to insert a fresh cutting blade unit into the cutting blade holder without any contact with the body. To this end, it is merely necessary to actuate the slide, which pushes the cutting blade unit out of the cartridge and into the blade receptacle of the cutting blade holder. The sterility and intactness of the cutting blade unit can thereby be preserved. No hand injuries are to be feared either. In addition, the coupling of the cartridge to the cutting blade holder and the actuation of the slide are not complicated and laborious activities for the surgeon or his assistants, particularly since the cartridge and the slide, owing to their size, are very much easier to grip and handle than the cutting blade unit on its own.

In order not only to be able to insert the cutting blade unit into the cutting blade holder, but also withdraw it again, it is advisable for the cutting blade unit and the slide to be designed for thrust- and traction-transmitting coupling to one another. To this end, a distal coupling head of the slide (distal here means remote from an actuating section at which the user grasps and operates the slide) can be engageable with a suitable coupling formation on the cutting blade unit by the key-and-lock principle.

In order to prevent the cutting blade unit from inappropriately falling out of the blade cartridge, the cartridge is assigned securing means which secure the cutting blade unit in the cartridge. The securing means can comprise a detachable securing element which is intended and designed for engagement in a recess of the cutting blade unit, which recess serves for coupling the cutting blade unit to a blade oscillating drive. Alternatively or additionally, it is possible for the securing means to comprise securing formations which ensure frictional securing of the cutting blade unit in the blade cartridge.

In a preferred embodiment, the slide is part of an actuating unit which is produced separately from and can be detachably coupled to the cartridge. The actuating unit can, for example, be a multiple-use product which does not have to be replaced after each use. In contrast, the cartridge can, for example, be provided as a single-use product which has to be completely replaced along with the cutting blade unit after each use. It is understood that, in an alternative embodiment, the cartridge and the actuating unit can be fixedly connected to one another, so that the entire fitting instrument including cartridge and actuating unit then always has to be replaced. In the case of a two-part design, the actuating unit can, for example, have a gripping sleeve which longitudinally displaceably receives the slide and which is formed in the region of a distal sleeve end for coupling to the cartridge.

The cutting blade of the cutting blade unit normally forms a cutting edge at a front blade edge, it being possible for the opposite, rear blade edge to form at least one aligning edge which can serve for aligning the cutting blade unit in the blade receptacle of the cutting blade holder. In order to protect the cutting edge and, if necessary, also the aligning edge—the latter ensures the required precise positioning of the cutting blade unit in the cutting blade holder—from possible damage when a cartridge is fitted by the manufacturer with a fresh cutting blade unit and also when the cutting blade unit is pushed out of the cartridge, the cartridge is preferably configured in such a way that, when the cutting blade unit is correctly received therein, the cutting edge and desirably the aligning edge of the cutting blade have a clearance all the way round. It can thus be ensured that the cutting edge and optionally the aligning edge do not come into contact with the walls of the cartridge on introduction of the cutting blade into and withdrawal from the cartridge. This ensures the intactness of these edges.

According to one embodiment, the cartridge can be configured to receive a single cutting blade unit, while in a modified embodiment it is designed to receive two or more cutting blade units. In the latter case, each cutting blade unit can be accommodated in a respective dedicated cartridge chamber of the blade cartridge, in which case the fitting device according to the invention can then also have a respective slide assigned to each cartridge chamber. It is, of course, not precluded that a plurality of cutting blade units can be received in a common cartridge chamber.

Protection is sought, in the context of the invention, furthermore for a blade cartridge for use in a fitting device of the two-part type described above with an actuating unit structurally separate from the cartridge. The cartridge is in this case fitted with at least one cutting blade unit. Protection is also sought for a cutting blade unit which is designed with at least one coupling formation which is intended and designed for thrust- and traction-transmitting coupling of the cutting blade unit to a distal coupling head of a slide of a fitting device according to the invention.

The invention is explained in more detail below with reference to the appended drawings, in which:

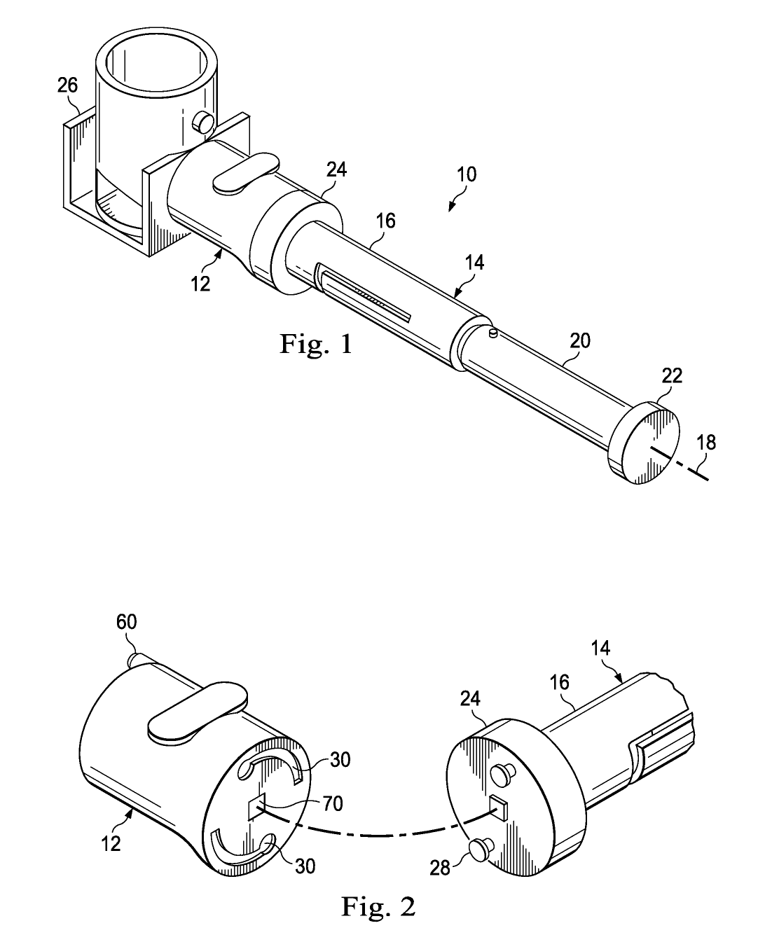

FIG. 1 illustrates, in perspective, an exemplary embodiment of a fitting device according to the invention in the ready-to-use state,

FIG. 2 illustrates, in perspective, a cartridge and a part of an actuating unit of the fitting device of FIG. 1,

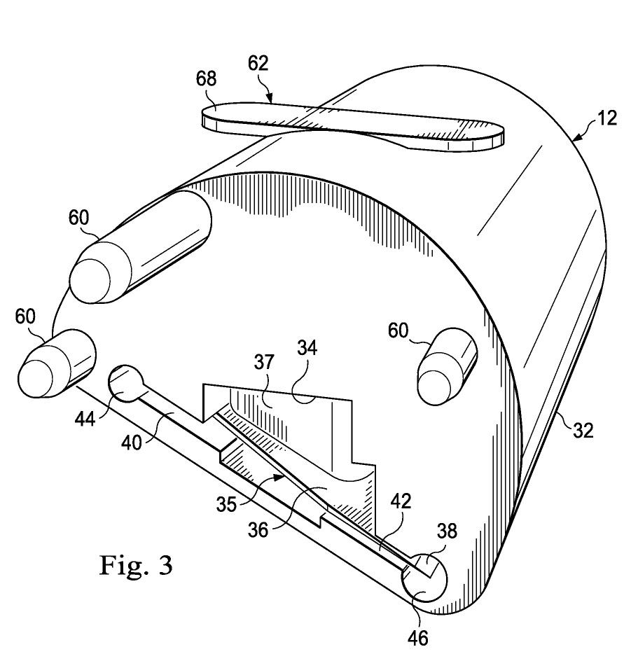

FIG. 3 is a perspective view of a cartridge end-side which, in the use position, faces a cutting head of a microkeratome,

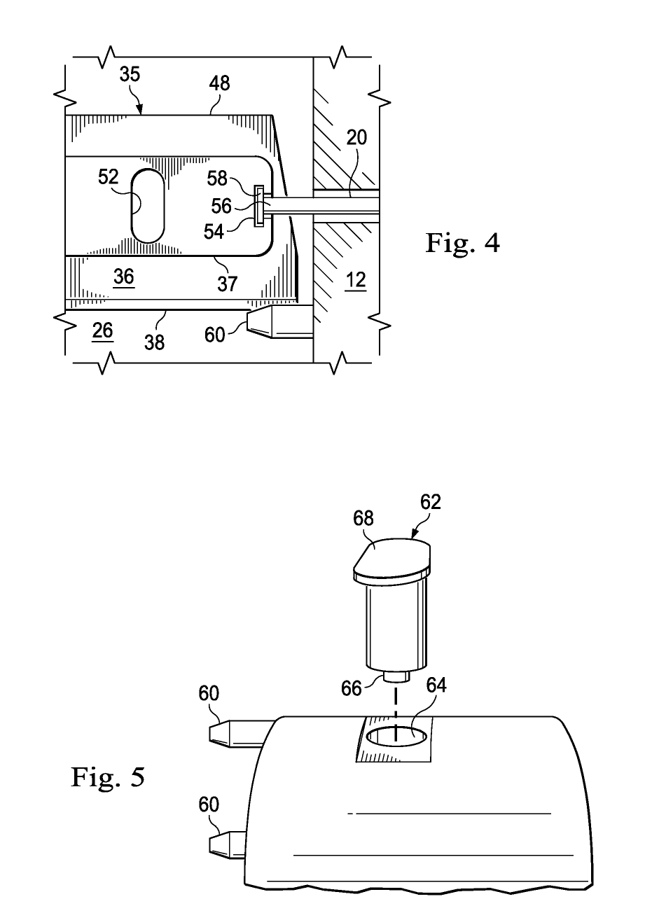

FIG. 4 illustrates, schematically, the engagement of an actuating rod of the actuating unit with a cutting blade unit,

FIG. 5 illustrates a securing element for securing the cutting blade unit in the cartridge,

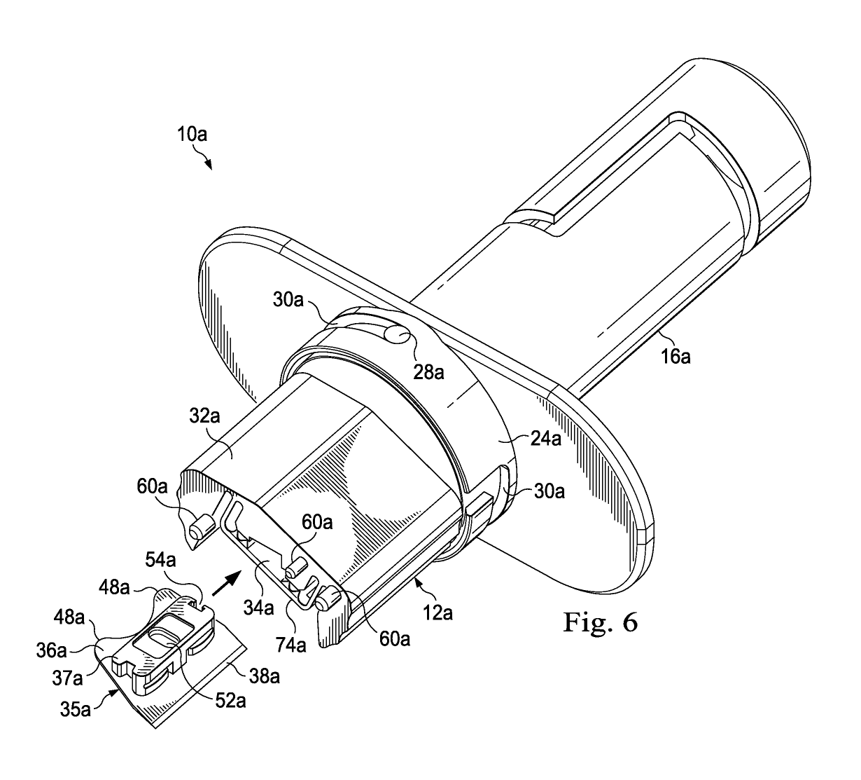

FIG. 6 illustrates, in perspective, a cartridge, a part of an actuating unit, and a cutting blade unit according to a further exemplary embodiment of the fitting device according to the invention, and

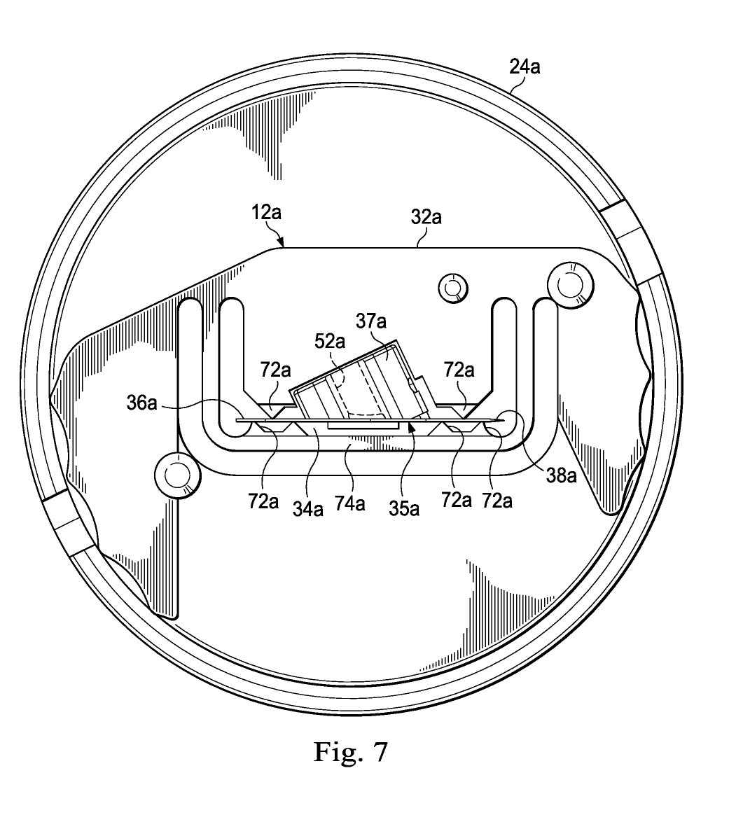

FIG. 7 is an axial plan view of a cutting-head-side end of the fitting device of FIG. 6.

The fitting device of FIG. 1 is denoted generally by 10. It comprises a cartridge 12 and an actuating unit 14 having an actuating rod 20 received in a gripping sleeve 16 in a manner longitudinally displaceable along a sleeve longitudinal axis 18. The actuating rod 20 forms a slide in accordance with the invention and has, at its proximal end, a gripping flange 22 which facilitates actuation. The gripping sleeve 16 has, at its distal end, a coupling section 24 which serves for coupling the actuating unit 14 to a first axial side of the cartridge 12. In the example of FIG. 1, the coupling section 24 is embodied as a radially projecting coupling flange 24.

The cartridge 12 is designed, on its opposite second (distal) axial side, for detachable coupling to a cutting blade holder (cutting head) 26 of a microkeratome for ophthalmic surgical treatments. The cutting head 26 has a receiving pocket (not illustrated specifically in the drawings) for a cutting blade unit, by means of which a flap can be cut from the surface of the cornea. The blade receiving pocket of the cutting head 26 is open towards the side of the cartridge 12, so that a new, unused cutting blade unit can be pushed out of the cartridge 12 into the blade receiving pocket of the cutting head by means of the actuating unit 14 and, after use, withdrawn from the pocket again.

Reference is now made additionally to FIG. 2. The actuating unit 14 and the cartridge 12 are produced structurally separately from one another and can be detachably connected to one another via cooperating coupling formations on the first axial side of the cartridge 12 and on the coupling section 24. In the illustrated example, these coupling formations form a turn-lock fastener, an arrangement of two or more axially projecting locking pins 28 with a cross-sectionally enlarged pin head being provided on one of the components: cartridge 12 and coupling section 24 (here on the coupling section 24). The other component (here the cartridge 12) has, assigned to each of the locking pins 28, a respective slot 30 which extends in the shape of a circular arc and is designed to be wide enough in the region of one slot end to permit the axial introduction of the associated locking pin 28 into the respective slot 30. On relative rotation of the cartridge 12 and the gripping sleeve 16, the locking pins 28 then slide in the slots 30, the cross-sectionally enlarged pin heads engaging axially behind the slot edges. It is thus ensured that the cartridge 12 and the gripping sleeve 16 are securely held together. The quality of the connection can be improved by designing the slot edges, behind which the pin heads engage, with elevations or ramp surfaces rising obliquely with respect to a plane normal to the axis, so that the turn-lock fastener acts in the manner of a threaded bayonet fastener, which, on relative rotation of the cartridge 12 and the gripping sleeve 16 in a suitable direction of rotation, causes these two components to be pressed against one another. Alternatively or additionally, the locking pins 28 can be secured at the end of their travel in the slots 30 by suitable latching means, so that an increased effort is required to move the locking pins 28 out of their latched position again and release the connection of the cartridge 12 to the gripping sleeve 16.

Reference is now made additionally to FIG. 3. As can be seen in this figure, the cartridge 12 has a cartridge housing 32, in which a cartridge chamber 34 is formed. The cartridge chamber 34 serves for the protected reception and holding of a cutting blade unit 35. The cutting blade unit 35 has a cutting blade 36 and a top part 37, preferably made of plastic and firmly connected to the blade 36, on one of the blade flat sides, see FIG. 4. At a front blade edge, the cutting blade 36 forms a cutting edge 38. The cartridge chamber 34 is designed cross-sectionally larger in a centre region, to be able to receive the top part 37 of the cutting blade unit 35. On both sides of the cross-sectionally enlarged centre region, the cartridge chamber 34 has sections narrowed in the shape of a slot, as indicated in FIG. 3 at 40 and 42. At their ends, the sections 40, 42 narrowed in the shape of a slot merge into cross-sectionally enlarged chamber end regions 44, 46, in which the cutting edge 38 and an aligning edge formed at the rear blade edge are positioned when the cutting blade unit 35 is correctly inserted into the cartridge chamber 34. In this position, the cutting edge 38 and the aligning edge (not discernible more clearly in FIG. 3) of the cutting blade 36 are spaced on all sides from the walls of the cartridge chamber 34, so that there is no risk of damage due to contact with the chamber walls. The cross-sectionally enlarged chamber end regions 44, 46 can be formed, for example, by bores which intersect the slot-like sections 40, 42.

For further explanation of the cutting blade 36, reference is now made additionally to FIG. 4. The aligning edge, which has just been mentioned, of the cutting blade 36 is denoted by 48 in this figure. The top part 37 contains a central, elongated slot 52 which, in a manner known per se, serves for the engagement of an eccentric pin of a drive shaft of an electric-motor drive unit (not illustrated specifically) for the cutting blade 36. The movement of the eccentric pin in the slot 52 sets the cutting blade 36 in lateral oscillation.

Formed laterally in the top part 37 is a coupling formation 54 which can be brought into thrust- and traction-transmitting engagement with a distal coupling head 56 of the actuating rod 20. The engagement between the coupling formation 54 of the cutting blade unit 35 and the coupling head 56 is positive and/or non-positive. In the illustrated example of FIG. 4, the coupling formation 54 is formed by an undercut T-slot in the top part 37, into which slot the coupling head 56 can be introduced. For this purpose, the coupling head 56 can bear a radially projecting collar formation 58 which is configured in such a way that, in a first rotational position of the actuating rod 20, the collar formation 58 can be introduced through the opening of the slot 54 into the latter and moved out of the latter, while in a second rotational position (for example rotated through 90°) of the actuating rod 20, the collar formation 58 is moved in front of the undercut surfaces of the slot 54. The latter situation is illustrated in FIG. 4. In this state, the actuating rod 20 is locked to the cutting blade unit 35. The undercut slot 54 and the collar formation 58 form a locking mechanism based on the key-and-lock principle, where the coupling head 56 forms the “key” and the slot 54 forms the “lock”. It is understood that other embodiments of a locking mechanism based on the key-and-lock principle are conceivable. It is further understood that the detachable coupling of the actuating rod 20 to the cutting blade unit 35 may also follow other mechanical or non-mechanical principles.

On its axial side facing the cutting head 26, the cartridge 12 bears an arrangement of positioning formations, by means of which the cartridge 12 can be positioned on the cutting head 26 in a position permitting the correct introduction of the cutting blade unit 35 into the blade receiving pocket. In the illustrated example, the positioning formations of the cartridge 12 are formed by axially projecting positioning pins 60 which, on joining together the cartridge 12 and the cutting head 26, engage in assigned positioning holes (not illustrated specifically) of the cutting head 26. By a suitable arrangement pattern of the positioning pins 60 or/and by variation of the thickness or/and length of the positioning pins, it can be ensured that the cartridge 12 can be correctly attached to the cutting head 26 only in a single, uniquely defined relative positioning. The coupling between the cartridge 12 and the cutting head 26 can be a loose coupling, but it can also be fixed by the action of suitable holding means. For example, the thickness of the positioning pins 60 can be matched to the diameter of the positioning holes provided on the cutting head 26 in such a way that a certain frictional engagement of the positioning pins 60 in the positioning holes occurs, which ensures cohesion of the cartridge 12 and the cutting head 26. Alternative possibilities for ensuring retention of the cartridge 12 on the cutting head 26 comprise, for example, the provision of suitable latching means.

Reference is now made furthermore to FIG. 5. In this figure, there can be seen a substantially pin-like securing element 62 which, for securing the cutting blade unit 35 in the cartridge 12, can be inserted into a bore 64 made in the cartridge housing 32 from an outer circumferential surface of the latter. The bore 64 opens into the cartridge chamber 34. At its leading end on correct insertion into the bore 64, the securing element 62 has a projecting securing peg 66 which is intended and designed to penetrate into the slot 52 of the blade top part 37 and thereby secure the cutting blade unit 35 against unintentionally falling out of the cartridge chamber 34. The securing element 62 can be expediently latched in the bore 64 by suitable latching means, so that it cannot unintentionally fall out of the bore 64. It has a projecting gripping section 68 which permits easy handling of the securing element 62.

In FIG. 2 it can also be seen that the cartridge 12 contains at its first axial side a through opening 70, through which the actuating rod 20 can pass, with its coupling head 56, and reach into the cartridge chamber 34.

The cartridge 12 can be provided by a manufacturing company as a single-use product, in which case it can already be fitted by the manufacturer with a cutting blade unit 35, the latter being securable in the cartridge 12, for example, by means of the securing element 62 or in another way. The actuating unit 14 can be manufactured and supplied as a separate constructional unit, in which case it can be intended as a single-use product, but also as a multiple-use product.

To assemble the fitting device, the gripping sleeve 16 is locked by its coupling section 24 to the cartridge 12. If the actuating rod 20 is completely removable from the gripping sleeve 16, the locking of the gripping sleeve 16 to the cartridge 12 can, if desired, be performed with the actuating rod 20 removed. It is, of course, also possible for the actuating rod 20 to be secured against complete removal from the gripping sleeve 16 by suitable stop means.

Subsequently, the cartridge 12 can be attached to the cutting head 26, and the coupling head 56 of the actuating rod 20 can be brought into engagement with the coupling formation 54 of the cutting blade unit 35 situated in the cartridge 12. After removal of the securing element 62—where present—the cutting blade unit 35 can then be pushed out of the cartridge chamber 34 and into the blade receiving pocket of the cutting head 26 by exerting pressure on the gripping flange 22 of the actuating rod 20. The sections 40, 42, narrowed in the manner of a slot, of the cartridge chamber 34 form a sliding guide for the cutting blade 36 in this process. The cutting blade 36 can thus be moved into the cutting head 26 in a defined guided manner.

After the cutting blade unit 35 has been introduced into the cutting head 26, the actuating rod 20 is detached from the cutting blade unit 35 again, for example by rotation. After that, the cartridge 12 can be removed from the cutting head 26 together with the actuating unit 14.

After a treatment with the cutting blade unit previously inserted into the cutting head, the cartridge 12 can be attached to the cutting head 26 again, together with the actuating unit 14, and the actuating rod 20 can be coupled to the cutting blade unit 35 situated in the cutting head 26. By withdrawing the actuating rod 20, the cutting blade unit 35 can be transferred back into the cartridge 12. The used cutting blade unit 35 can be secured in the cartridge 12 again, for example by means of the securing element 62. The cartridge 12 can then be disposed of with the cutting blade unit 35 situated therein.

The various components of the fitting device 10 can be produced from materials such as plastic or metal. In the case of single-use products, plastic is recommended because it can be produced more easily. In general, care will have to be taken to use materials that are readily sterilisable.

In FIGS. 6 and 7, identical or identically acting components are denoted with the same reference numerals as before, but supplemented by a small letter. In the exemplary embodiment shown in these figures, the cutting blade unit 35a cannot be secured in the cartridge chamber 34a by a securing pin attachable separately to the cartridge 12a. Instead, in the cartridge chamber 34a there are provided securing formations which hold the cutting blade unit 35a frictionally in the cartridge chamber 34a and thus prevent the cutting blade unit 35a from unintentionally falling out of the cartridge chamber 34a. For a detailed explanation of these securing formations, reference is made to FIG. 7 in particular. In this figure there can be seen a plurality of securing webs 72a which are provided in the cartridge chamber 34a on both sides of the blade plane of the cutting blade 36a. In the example shown, the securing webs 72a are designed in an approximately tapering manner. A cross-sectionally roundish contour of the securing webs 72a is, of course, also possible. The securing webs 72a are arranged in the cartridge chamber 34a in such a way that they hold the cutting blade 36a and thus the cutting blade unit 35a between them when the cutting blade unit 35a is inserted into the cartridge chamber 34a.

In FIG. 7 there can also be seen the clearance which exists around the cutting blade 38a and the rear blade edge of the cutting blade 36a when the cutting blade unit 35a is inserted into the cartridge chamber 34a. Overall, the cartridge chamber 34a in the exemplary embodiment of FIGS. 6 and 7 has an approximately U-shaped profile, there being provided in the region of the U-web the cross-sectionally widened section, already mentioned before in connection with the exemplary embodiment of FIGS. 1 to 5, for receiving the top part 37a of the cutting blade unit 35a. On the flat side, remote from the top part, of the cutting blade 36a, the cartridge chamber 34a is bounded by a boundary wall 74a running in the manner of a stirrup. This boundary wall 74a can also be clearly seen in FIG. 6.

The securing webs 72a can be formed, for example, integrally with the material of the cartridge housing 32a. The cartridge housing 32a for its part can be of one or more parts.

The securing webs 72a can extend, in the blade transverse direction, i.e. in the direction perpendicular to the plane of the drawing of FIG. 7, over a length corresponding at least approximately to the width of the cutting blade 36a. For the aim of securing the cutting blade unit 35a in the cartridge chamber 34a, it may also be sufficient to design the securing webs 72a as comparatively short web segments which are substantially shorter than the width of the cutting blade 36a. Furthermore, instead of elongated securing webs or ribs, it is conceivable to provide a plurality of knob-like securing projections in the cartridge chamber 34a on both sides of the cutting blade 36a. Such securing knobs can likewise have an approximately tapering or a roundish contour.

In FIG. 6 it can furthermore be seen that the cutting blade 36a does not form a single straight aligning edge at its rear blade edge, but is designed with two bearing regions 48a which are provided spaced apart from one another and between which the rear blade edge is set back. The bearing regions 48a are of roundish design in the example shown, so that they can form an approximately point contact with an abutment surface provided in the cutting head of the cutting instrument.

In the exemplary embodiment of FIGS. 6 and 7, a further difference from the exemplary embodiment of FIGS. 1 to 5 is that the locking pins 28a are provided on the cartridge 12a, while the slots 30a are formed in the coupling section 24a of the gripping sleeve 16a. The coupling section 24a is formed here as a cylindrical section which encompasses the cartridge 12a radially on the outside. Accordingly, the slots 30a are formed in the cylinder wall of the coupling section 24a, while the locking pins 28a project radially from the outer circumference of the cartridge 12a. Here, too, by means of an oblique course of the slots 30a with respect to the circumferential direction, it is possible to achieve a threaded effect which can ensure that the gripping sleeve 16a and the cartridge 12a are axially pressed against on another.

What is claimed is:

1. Device for fitting a cutting blade holder of a microsurgical cutting instrument, in particular a cutting instrument for use in refractive eye treatments, with a cutting blade unit, comprising:
a blade cartridge which can be detachably coupled to the cutting blade holder and in which at least one cutting blade unit is received;
a slide which is or can be held slidably relative to the cartridge and by means of which the cutting blade unit can be pushed out of the blade cartridge into a blade receptacle of the cutting blade holder.

2. Device according to claim 1, characterised in that the cutting blade unit and the slide are designed for thrust- and traction-transmitting coupling to one another.

3. Device according to claim 1, characterised in that the cartridge is assigned securing means which secure the cutting blade unit in the cartridge.

4. Device according to claim 3, characterised in that the securing means comprise a detachable securing element which is intended and designed for engagement in a recess of the cutting blade unit, which recess serves for coupling the cutting blade unit to a blade oscillating drive.

5. Device according to claim 3, characterised in that the securing means comprise securing formations which ensure frictional securing of the cutting blade unit in the blade cartridge.

6. Device according to claim 1, characterised in that the slide is part of an actuating unit which is detachably coupled to the blade cartridge.

7. Device according to claim 6, characterised in that the actuating unit has a gripping sleeve which longitudinally displaceably receives the slide and which is formed in the region of a distal sleeve end for coupling to the blade cartridge.

8. Device according to one of the preceding claims, characterised in that the cutting blade unit has a cutting blade with a cutting edge at one blade edge and with at least one aligning edge at an opposite blade edge, the aligning edge serving for aligning the cutting blade unit in the blade receptacle of the cutting blade holder, and in that the blade cartridge is configured in such a way that, when the cutting blade unit is correctly received therein, the cutting edge and desirably the aligning edge of the cutting blade have a clearance all the way round.

9. Device according to claim 6, wherein the blade cartridge is fitted with at least one cutting blade unit.

10. Cutting blade unit for use with an inserter rod having a distal head with a first coupling formation configured for engagement with a blade unit to transmit both thrust and traction forces, the cutting blade unit comprising: a body member with at least one second coupling formation which is intended and designed for engagement with the first coupling formation of the inserter rod thereby thrust- and traction-transmitting coupling to a distal coupling head of the slide inserter.

11. A cutting blade unit inserter for securely delivering a cutting blade unit to a cutting blade holder of a microsurgical cutting instrument, the cutting blade unit inserter, comprising:
a body having a passage for retaining a least a portion of a cutting blade unit, the passage having a proximal end and an opposite distal end;
a first portion of a coupling assembly formed adjacent the distal end, the first portion of the coupling assembly configured for engagement with a cooperating second portion of the coupling assembly formed on the cutting blade holder; and
an actuator having a distal end, said distal end configured to extend along at least a portion of said passage from said proximal end toward said distal end, wherein with said first portion of the coupling assembly engaged with said second portion of said couple assembly on the cutting blade holder, movement of said actuator along said passage urges the cutting blade unit positioned in said passage out of said distal end of said passage and into the cutting blade holder.

12. The apparatus of claim 11, wherein said first portion of the coupling assembly includes at least one pin.

13. The apparatus of claim 11, further including an actuating unit slidably receiving said actuator.

14. The apparatus of claim 13, wherein said body further includes a third portion of a second coupling assembly formed adjacent the proximal end of the passage, the third portion configured for engagement with a fourth portion of the second coupling formed on said actuating unit.

15. The apparatus of claim 11, wherein said actuator comprises a rod having a distal end, the distal end including at least one radial projection configured for mating engagement with a cutting blade unit.

16. The apparatus of claim 15, wherein said rod is received in an actuating unit and rotatable within said unit between a first radial position with said radial projection in a locked position and a second radial position with said radial projection in an unlocked position.
